(19) United States
(12) Patent Application Publication
(10) Pub. No.: US 2006/0101594 A1
(43) Pub. Date: May 18, 2006

(54) Phthalimidyl-azo dyes, process for their preparation and their use

(76) Inventors: Antoine Clement, Basel (CH); Alfons Arquint, Basel (CH); Urs Lauk, Basel (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/533,010

(22) PCT Filed: Nov. 4, 2003
(86) PCT No.: PCT/EP03/12282

§ 371 Date: Apr. 28, 2005

(30) Foreign Application Priority Data
Nov. 11, 2002 (EP) 02405967.7

(51) Int. Cl. A61K 8/00

(52) U.S. Cl. 8/405

(57) ABSTRACT

The present invention relates to dyes of formula (I) wherein R₁, is 2-methoxyethyl, 2-ethoxyethyl or 2-(2-ethoxyethoxy)ethyl, R₂ is C_{1,}-C₆alkyl which is unsubstituted or substituted by one or more hydroxy groups, halogen atoms, C_{1,}-C₄alkoxy groups, C₂-C₈alkoxyalkoxy groups, C_{1,}-C₄alkyl-COO— groups or C_{1,}-C₄alkyl-OCO— groups, R₃ is methyl, hydroxy, halogen, —NH—SO₂—R₅ or —NH—CO—R₆, wherein R₅ is methyl or ethyl and R₆ is methyl, ethyl, methoxymethyl or 2-methoxyethyl, and R₄ is ethyl or n-propyl, and also to a process for their preparation and to their use in the dyeing or printing of semi-synthetic and, especially, synthetic hydrophobic fibre materials, more especially textile materials.

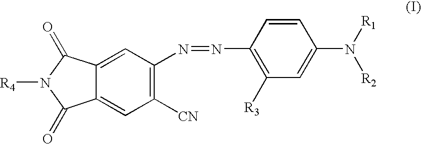

The present invention relates to disperse dyes having an N-alkyl-phthalimide diazo component and an aniline coupling component, to a process for the preparation of those dyes and to their use in the dyeing or printing of semi-synthetic and, especially, synthetic hydrophobic fibre materials, more especially textile materials.

Azo disperse dyes having an N-alkyl-phthalimide diazo component and an aniline coupling component have been known for a long time and are used in the dyeing of hydrophobic fibre materials. It has been shown, however, that the dyeings or prints obtained with the dyes known at present do not satisfy today's demands in all cases, especially in respect of fastness to washing and perspiration. There is therefore a need for new dyes which exhibit especially good fastness properties to washing.

It has now been found, surprisingly, that the dyes according to the invention meet the criteria given above to a considerable degree.

The present invention accordingly relates to disperse dyes which yield dyeings that have high fastness to washing and perspiration, and which additionally possess good build-up both in the exhaust and thermosol process and in textile printing. The dyes are also suitable for discharge printing.

The dyes according to the invention correspond to formula 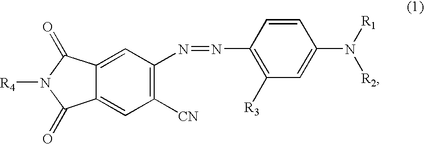
wherein R₁ is 2-methoxyethyl, 2-ethoxyethyl or 2-(2-ethoxyethoxy)ethyl, R₂ is C₁-C₆alkyl which is unsubstituted or substituted by one or more hydroxy groups, halogen atoms, C₁-C₄alkoxy groups, C₂-C₈alkoxyalkoxy groups, C₁-C₄alkyl-COO— groups or C₁-C₄alkyl-OCO— groups,

R₃ is methyl, hydroxy, halogen, —NH—SO₂—R₅ or —NH—CO—R₆, wherein R₅ is methyl or ethyl and R₆ is methyl, ethyl, methoxymethyl or 2-methoxyethyl, and R₄ is ethyl or n-propyl.

C₁-C₆Alkyl as R₂ may be, for example, methyl, ethyl, n-propyl, isopropyl, n-butyl, isobutyl, sec-butyl, tert-butyl, n-pentyl, neopentyl or n-hexyl.

Halogen as R₃ is fluorine, bromine or, preferably, chlorine.

R₁ and R₂ preferably have the same meaning.

R₁ is preferably 2-methoxyethyl.

R₃ is preferably —NH—CO—R₆.

R₆ is preferably methyl.

R₄ is preferably ethyl.

Special preference is given to dyes of formulae (1a), (1b) and (1c) 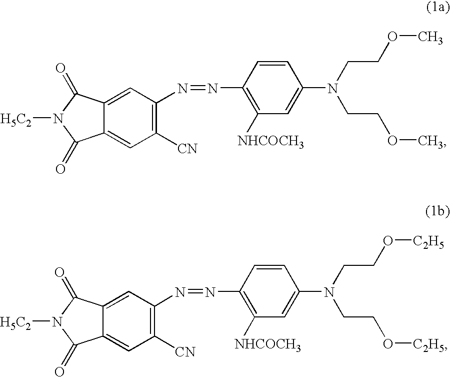 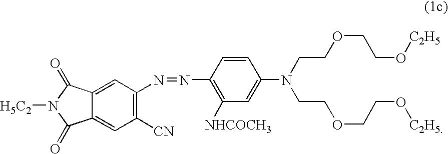
The present invention relates also to a process for the preparation of the dyes of formula (1), in which a phthalimide of formula 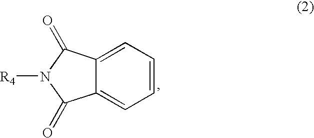
wherein R₄ is as defined above, is nitrated in the acidic range, as described, for example, on page 459 in Organic Synthesis, Collective Volume 2, (a Revised Edition of Annual Volumes X-XIX), J. Wiley & Sons, the resulting nitro compound is then alkylated, for example according to a method indicated in Journal of Organic Chemistry 32 (1967) on page 1923, paragraph 3, and converted, for example by means of reductive treatment as described in Bull. Soc. Chim. de France 1957 on page 569, into an intermediate of formula 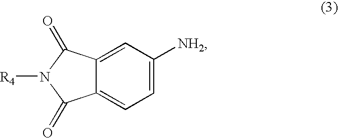
the intermediate of formula (3) is monohalogenated according to generally known methods in an acidic medium, then diazotised and coupled to a compound of formula (4) 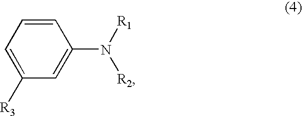
wherein R₁, R₂ and R₃ are as defined above, and then the halogen substituent is replaced by cyano.

The halogenation is carried out, for example, by reacting the compound of formula (3) first in acetic acid with sodium acetate and then, in the same medium, with bromine to form the corresponding monobromine compound.

The diazotisation is also carried out in a manner known per se, for example with sodium nitrite in an acidic, e.g. hydrochloric-acid-containing or sulfuric-acid-containing, aqueous medium. The diazotisation may, however, also be carried out using other diazotisation agents, for example using nitrosylsulfuric acid. In the diazotisation, an additional acid may be present in the reaction medium, e.g. phosphoric acid, sulfuric acid, acetic acid, propionic acid or hydrochloric acid or a mixture of such acids, e.g. a mixture of propionic acid and acetic acid. The diazotisation is advantageously carried out at temperatures of from −10 to 30° C., for example from −10° C. to room temperature.

The coupling of the diazotised compound to the coupling component of formula (4) is likewise effected in known manner, for example in an acidic, aqueous or aqueous-organic, medium, advantageously at temperatures of from −10 to 30° C., especially below 10° C. Examples of acids used are hydrochloric acid, acetic acid, propionic acid, sulfuric acid and phosphoric acid.

The reaction to the final dyes is carried out by cyano replacement in the monohalogen compound according to methods known per se, for example using CuCN or mixtures of CuCN and alkali metal cyanide in solvents, for example dimethylformamide, dimethyl sulfoxide, dimethylacetamide, N-methylpyrrolidone, sulfolane or pyridine.

The diazo components and the coupling components of formula (4) are known or can be prepared in a manner known per se.

The present invention relates also to dye mixtures comprising at least two structurally different azo dyes of formula (1).

Dye mixtures that comprise two structurally different azo dyes of formula (1) are preferred.

The dye mixtures according to the invention comprising at least two structurally different azo dyes of formula (1) can be prepared, for example, by simply mixing the individual dyes.

The amount of the individual dyes in the dye mixtures according to the invention can vary within a wide range of, for example, from 95:5 to 5:95 parts by weight, especially from 70:30 to 30:70 parts by weight, more especially from 55:45 to 45:55 parts by weight of the individual dyes in a dye mixture comprising two azo dyes according to the invention.

The dyes and dye mixtures according to the invention can be used in the dyeing or printing of semi-synthetic and, especially, synthetic hydrophobic fibre materials, more especially textile materials. Textile materials composed of blends that contain such semi-synthetic or synthetic hydrophobic fibre materials can likewise be dyed or printed using the dyes or dye mixtures according to the invention.

Suitable semi-synthetic fibre materials are especially cellulose 2½-acetate and cellulose triacetate.

Synthetic hydrophobic fibre materials consist especially of linear, aromatic polyesters, for example polyesters of terephthalic acid and glycols, especially ethylene glycol, or condensation products of terephthalic acid and 1,4-bis(hydroxymethyl)cyclohexane; of polycarbonates, e.g. polycarbonates of α,α-dimethyl-4,4-dihydroxy-diphenylmethane and phosgene, or of fibres based on polyvinyl chloride and on polyamide.

The application of the dyes and dye mixtures according to the invention to the fibre materials is effected in accordance with known dyeing procedures. For example, polyester fibre materials are dyed in the exhaust process from an aqueous dispersion in the presence of customary anionic or non-ionic dispersants and optionally customary swelling agents (carriers) at temperatures of from 80 to 140° C. Cellulose 2½-acetate is dyed preferably at from 65 to 85° C. and cellulose triacetate at temperatures of from 65 to 115° C.

The dyes and dye mixtures according to the invention will not colour wool and cotton present in the dyebath at the same time or will colour such materials only slightly (very good reservation), so that they can also be used satisfactorily in the dyeing of polyester/wool and polyester/cellulosic fibre blend fabrics.

The dyes and dye mixtures according to the invention are suitable for dyeing in accordance with the thermosol process, in the exhaust process and for printing processes.

The said fibre materials can be in a variety of processing forms, e.g. in the form of fibres, yarns or non-wovens, in the form of woven fabrics or knitted fabrics.

It is advantageous to convert the dyes or dye mixtures according to the invention into a dye preparation prior to use. For this purpose, the dye is ground so that its particle size is on average from 0.1 to 10 microns. The grinding can be carried out in the presence of dispersants. For example, the dried dye is ground with a dispersant or is kneaded into paste form with a dispersant and then dried in vacuo or by atomisation. The preparations so obtained can be used, after the addition of water, to prepare printing pastes and dyebaths.

For printing, customary thickeners will be used, e.g. modified or unmodified natural products, for example alginates, British gum, gum arabic, crystal gum, locust bean flour, tragacanth, carboxymethylcellulose, hydroxyethylcellulose, starch or synthetic products, for example polyacrylamides, polyacrylic acid or copolymers thereof or polyvinyl alcohols.

The dyes and dye mixtures according to the invention impart to the mentioned materials, especially to polyester materials, level colour shades having very good in-use fastness properties, such as, especially, good fastness to light, fastness to thermofixing, to pleating, to chlorine and to wetting, such as fastness to water, perspiration and washing; the finished dyeings are also distinguished by very good fastness to rubbing. Special mention is to be made of the good fastness to perspiration and, especially, to washing of the resulting dyeings.

The dyes and dye mixtures according to the invention can also be used satisfactorily in the preparation of mixed shades together with other dyes.

In addition, the dyes and dye mixtures according to the invention are also highly suitable for the dyeing of hydrophobic fibre materials from supercritical CO₂.

The present invention relates also to the above-mentioned use of the dyes and dye mixtures according to the invention, and to a method of dyeing or printing semi-synthetic or synthetic hydrophobic fibre materials, especially textile materials, which method comprises applying a dye according to the invention to the mentioned materials or incorporating it therein. The mentioned hydrophobic fibre materials are especially textile polyester materials. Further substrates which can be treated by the method according to the invention, as well as preferred process conditions, are to be found hereinbefore in the more detailed explanation of the use of the dyes according to the invention.

The invention relates also to hydrophobic fibre materials, especially polyester textile materials, which have been dyed or printed by the mentioned method. The dyes according to the invention are also suitable for modern recording processes, e.g. thermotransfer printing.

The Examples which follow serve to illustrate the invention. In the Examples, parts are parts by weight and percent are percent by weight, unless indicated otherwise. Temperatures are given in degrees Celsius. The relationship between parts by weight and parts by volume is the same as that between grams and cubic centimetres.

### I. PREPARATION EXAMPLES

### Example I.1

A. 5.5 ml of 96% sulfuric acid are placed in a laboratory reaction apparatus. 1.3 ml of ice-water are added dropwise with stirring and while cooling with an ice bath. Then, at an internal temperature of 25° C., 2.7 g of the compound of formula 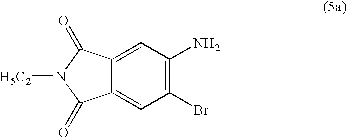
are introduced. The suspension so obtained is stirred for 10 minutes. 1.85 ml of 40% nitrosylsulfuric acid are then added dropwise in the course of 10 minutes, while cooling at 18-20° C., and stirring is carried out for 2 hours at that temperature.

1 ml of 32% hydrochloric acid in 60 ml of ice-water is introduced into a 250 ml laboratory reaction apparatus. A solution of 6.5 g of a 48% aqueous solution of the compound of formula (4a) 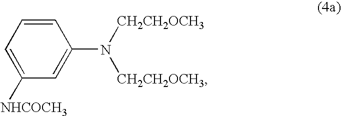
diluted with 25 ml of acetic acid, is added with stirring.

The solution of the diazonium salt is then added in the course of 10 minutes; at the same time, about 50 g of ice are introduced so that the internal temperature is 0-5° C.

The red suspension so obtained is stirred for 1 hour at 0-5° C., filtered off with suction, washed with deionised water and dried.

4.8 g (88% of the theoretical yield) of the compound of formula 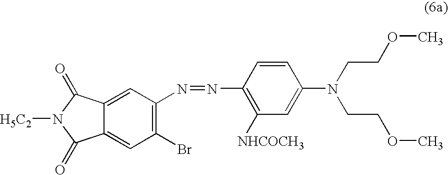
are obtained.

Melting point: 175-178° C.

Appearance: yellowish brown-red

B.2.23 g of the compound of formula (6a) and 10 ml of dimethyl sulfoxide are placed in a 100 ml laboratory reaction apparatus and stirred at room temperature (RT) for 10 minutes. 0.17 g of sodium cyanide and 0.30 g of copper(I) cyanide are then introduced, and the mixture is stirred for 10 minutes at RT. The temperature is then raised to 70-75° C. The reaction mixture is stirred for 2 hours at that temperature. After cooling to 60° C. and the dropwise addition of 5 ml of water, the suspension is filtered off with suction, washed with warm water (50° C.) and dried.

1.8 g (73% of the theoretical yield) of the azo dye of formula 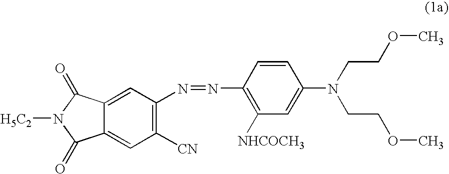
are obtained.

Melting point: 200-203° C.

Appearance: violet

The following dyes, which are likewise suitable for the dyeing of semi-synthetic or synthetic hydrophobic fibre materials, can be prepared analogously to Example I.1 (Table 1):

TABLE 1

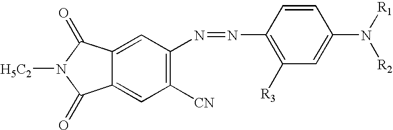

| R₁ | R₂ | R₃ |
|---|---|---|
| —CH₂CH₂OCH₃ | —CH₂CH₂OCH₃ | —CH₃ |
| ″ | —C₂H₅ | ″ |
| ″ | —CH₂CH₂CH₃ | ″ |
| ″ | —CH₂COOCH₃ | ″ |
| ″ | —CH₂COOC₂H₅ | ″ |
| ″ | —CH(CH₃)—COOCH₃ | ″ |
| ″ | —CH(CH₃)—COOC₂H₅ | ″ |
| ″ | —CH₂CH₂COOCH₃ | ″ |
| ″ | —CH₂CH₂COOC₂H₅ | ″ |
| ″ | —CH₂CH₂O—CO—CH₃ | ″ |
| ″ | —CH₂CH₂O—CO—C₂H₅ | ″ |
| ″ | Benzyl | ″ |
| ″ | —C₂H₅ | —NH—CO—CH₃ |
| ″ | —CH₂CH₂CH₃ | ″ |
| ″ | —CH₂COOCH₃ | ″ |
| ″ | —CH₂COOC₂H₅ | ″ |
| ″ | —CH(CH₃)—COOCH₃ | ″ |
| ″ | —CH(CH₃)—COOC₂H₅ | ″ |
| ″ | —CH₂CH₂COOCH₃ | ″ |
| ″ | —CH₂CH₂COOC₂H₅ | ″ |
| ″ | —CH₂CH₂O—CO—CH₃ | ″ |
| ″ | —CH₂CH₂O—CO—C₂H₅ | ″ |
| ″ | Benzyl | ″ |
| ″ | —CH₂CH₂OCH₃ | —NH—CO—C₂H₅ |
| ″ | —C₂H₅ | ″ |
| ″ | —CH₂CH₂CH₃ | ″ |
| ″ | —CH₂COOCH₃ | ″ |
| ″ | —CH₂COOC₂H₅ | ″ |
| ″ | —CH(CH₃)—COOCH₃ | ″ |
| ″ | —CH(CH₃)—COOC₂H₅ | ″ |
| ″ | —CH₂CH₂COOCH₃ | ″ |
| ″ | —CH₂CH₂COOC₂H₅ | ″ |
| ″ | —CH₂CH₂O—CO—CH₃ | ″ |
| ″ | —CH₂CH₂O—CO—C₂H₅ | ″ |
| ″ | Benzyl | ″ |
| ″ | —CH₂CH₂OCH₃ | —NH—SO₂—CH₃ |
| ″ | —C₂H₅ | ″ |
| ″ | —CH₂CH₂CH₃ | ″ |
| ″ | —CH₂COOCH₃ | ″ |
| ″ | —CH₂COOC₂H₅ | ″ |
| ″ | —CH(CH₃)—COOCH₃ | ″ |
| ″ | —CH(CH₃)—COOC₂H₅ | ″ |
| ″ | —CH₂CH₂COOCH₃ | ″ |
| ″ | —CH₂CH₂COOC₂H₅ | ″ |
| ″ | —CH₂CH₂O—CO—CH₃ | ″ |
| ″ | —CH₂CH₂O—CO—C₂H₅ | ″ |
| ″ | Benzyl | ″ |
| ″ | —CH₂CH₂OCH₃ | —NH—SO₂—CH₃ |
| ″ | —C₂H₅ | ″ |
| ″ | —CH₂CH₂CH₃ | ″ |
| ″ | —CH₂COOCH₃ | ″ |
| ″ | —CH₂COOC₂H₅ | ″ |
| ″ | —CH(CH₃)—COOCH₃ | ″ |
| ″ | —CH(CH₃)—COOC₂H₅ | ″ |
| ″ | —CH₂CH₂COOCH₃ | ″ |
| ″ | —CH₂CH₂COOC₂H₅ | ″ |
| ″ | —CH₂CH₂O—CO—CH₃ | ″ |
| ″ | —CH₂CH₂O—CO—C₂H₅ | ″ |
| ″ | Benzyl | ″ |
| ″ | —CH₂CH₂OCH₃ | —NH—CO—CH₂OCH₃ |
| ″ | —C₂H₅ | ″ |
| ″ | —CH₂CH₂CH₃ | ″ |
| ″ | —CH₂COOCH₃ | ″ |
| ″ | —CH₂COOC₂H₅ | ″ |
| ″ | —CH(CH₃)—COOCH₃ | ″ |
| ″ | —CH(CH₃)—COOC₂H₅ | ″ |
| ″ | —CH₂CH₂COOCH₃ | ″ |
| ″ | —CH₂CH₂COOC₂H₅ | ″ |
| ″ | —CH₂CH₂O—CO—CH₃ | ″ |
| ″ | —CH₂CH₂O—CO—C₂H₅ | ″ |
| ″ | Benzyl | ″ |
| ″ | —CH₂CH₂OCH₃ | —OH |
| ″ | —C₂H₅ | ″ |
| ″ | —CH₂CH₂CH₃ | ″ |
| ″ | —CH₂COOCH₃ | ″ |
| ″ | —CH₂COOC₂H₅ | ″ |
| ″ | —CH(CH₃)—COOCH₃ | ″ |
| ″ | —CH(CH₃)—COOC₂H₅ | ″ |
| ″ | —CH₂CH₂COOCH₃ | ″ |
| ″ | —CH₂CH₂COOC₂H₅ | ″ |
| ″ | —CH₂CH₂O—CO—CH₃ | ″ |
| ″ | —CH₂CH₂O—CO—C₂H₅ | ″ |
| ″ | Benzyl | ″ |

### II. APPLICATION EXAMPLES

### Example II.1

1 part by weight of the dye of formula 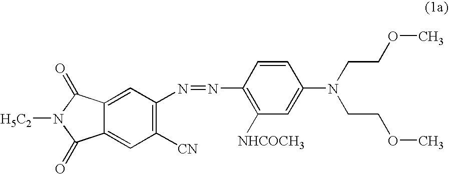
is ground in a sand mill together with 17 parts by weight of water and 2 parts by weight of a commercially available dispersant of the dinaphthylmethanedisulfonate type and converted into a 5% aqueous dispersion.

Using that formulation, a 1% dyeing (based on dye and substrate) is produced on polyester fabric by the high-temperature exhaust process at 130° C. and is reduction cleared. The violet dyeing so obtained has very good in-use fastness properties, especially excellent fastness to washing.

The same good fastness properties can be achieved when polyester fabric is dyed by the thermosol process (10 g/l of dye, liquor pick-up 50%, fixing temperature 210° C.).

1. A dye of formula 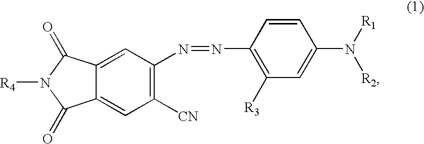
wherein R₁ is 2-methoxyethyl, 2-ethoxyethyl or 2-(2-ethoxyethoxy)ethyl,
R₂ is C₁-C₆alkyl which is unsubstituted or substituted by one or more hydroxy groups, halogen atoms, C₁-C₄alkoxy groups, C₂-C₈alkoxyalkoxy groups, C₁-C₄alkyl-COO— groups or C₁-C₄alkyl-OCO— groups R₃ is methyl, hydroxy, halogen, —NH—SO₂—R₅ or —NH—CO—R₆, wherein R₅ is methyl or ethyl and R₆ is methyl, ethyl, methoxymethyl or 2-methoxyethyl, and
R₄ is ethyl or n-propyl.

2. A dye according to claim 1, wherein R₁ and R₂ are the same.

3. A dye according to claim 1, wherein R₁ is 2-methoxyethyl.

4. A dye according to claim 1, wherein R₃ is —NH—CO—R₆.

5. A dye according to claim 4, wherein R₆ is methyl.

6. A dye according to claim 1, wherein R₄ is ethyl.

7. A dye of formula (1a), (1b) or (1c) according to claim 1

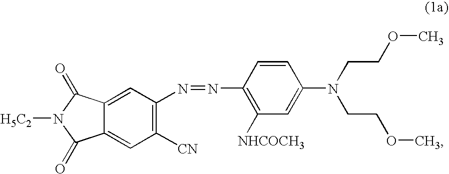

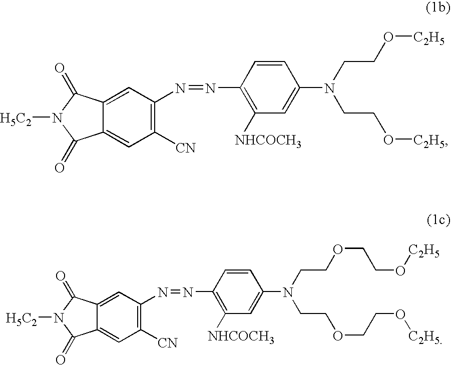

8. A process for the preparation of a dye of formula (1) according to claim 1, in which a phthalimide of formula 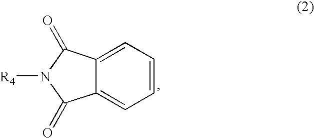
wherein R₄ is as defined in claim 1,
is nitrated in the acidic range, the resulting nitro compound is then alkylated and converted by means of reductive treatment into an intermediate of formula 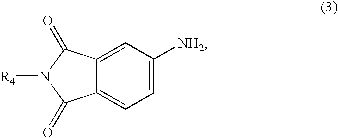
the intermediate of formula (3) is monohalogenated in an acidic medium and then diazotised, coupled to a compound of formula (4) 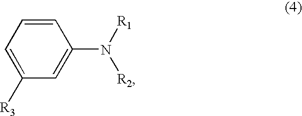
wherein R₁, R₂ and R₃ are as defined in claim 1, and then the halogen substituent is replaced by cyano.

9. A dye mixture comprising at least two structurally different dyes of formula (1) according to claim 1.

10. A method of dyeing or printing a semi-synthetic or synthetic hydrophobic fibre material, in which a dye of formula (1) according to claim 1 is applied to the mentioned material or incorporated therein.

11. (canceled)

12. A semi-synthetic or, synthetic hydrophobic fibre material, which has been dyed or printed by the process according to claim 10.

13. A method of dyeing or printing a semi-synthetic or synthetic hydrophobic fibre material, in which a dye mixture according to claim 9 is applied to the mentioned material or incorporated therein.

14. A semi-synthetic or synthetic hydrophobic textile fibre material which has been dyed or printed by the process according to claim 10.
